(19) United States
(12) Patent Application Publication
(10) Pub. No.: US 2025/0281473 A1
(43) Pub. Date: Sep. 11, 2025

(54) CDK Inhibitors And Their Use As Pharmaceuticals

(71) Applicant: PRELUDE THERAPEUTICS INCORPORATED, Wilimington, DE (US)

(72) Inventors: Krishna Vaddi, Chadds Ford, PA (US); Norman Fultang, Philadelphia, PA (US); Neha Bhagwat, Springfield, PA (US); Peggy Scherle, Wayne, PA (US)

(21) Appl. No.: 19/073,429

(22) Filed: Mar. 7, 2025

Related U.S. Application Data
(60) Provisional application No. 63/562,769, filed on Mar. 8, 2024.

(51) Int. Cl. A61K 31/4439; A61K 31/415; A61K 31/496; A61K 31/4985; A61K 31/519; A61P 35/00; A61P 35/02

(52) U.S. Cl. CPC A61K 31/4439; A61K 31/415; A61K 31/496; A61K 31/4985; A61K 31/519; A61P 35/00; A61P 35/02

(57) ABSTRACT

Methods of treating cancer in a subject in need thereof, comprising administering to the subject a treatment regimen comprising a compound of Formula I, as defined herein, or a pharmaceutically acceptable salt thereof, and a second compound that is a BTK inhibitor or a BCL2 inhibitor.

### CROSS-REFERENCE TO RELATED APPLICATION

This application claims the benefit of U.S. Provisional Application No. 63/562,769, filed Mar. 8, 2024, the entirety of which is incorporated by reference herein.

### TECHNICAL FIELD

The present invention relates to the field of pharmaceutical compounds for the treatment of cancer. In particular, the disclosure pertains to methods of treating cancer comprising administering to a subject a treatment regimen comprising a compound of Formula (I) and a BTK inhibitor or BCL2 inhibitor.

### BACKGROUND

Cyclin-dependent kinase 9 (CDK9) is a master regulator of transcription that modulates transcription elongation via phosphorylation of RNA polymerase II. Short-term inhibition of CDK9 depletes short-lived transcripts and labile proteins such as MCL1, BFL1 and MYC to promote cancer cell death.

Selective CDK9 inhibition may be a promising approach to treat transcription-addicted cancers that are dependent on oncogenic drivers with a short half-life, such as the oncogenes myelocytomatosis (MYC), myelobastosis (MYB), and myeloid leukemia cell differentiation protein (MCL1).

The compound of Formula (I) is a CDK9 inhibitor having the structure shown below:

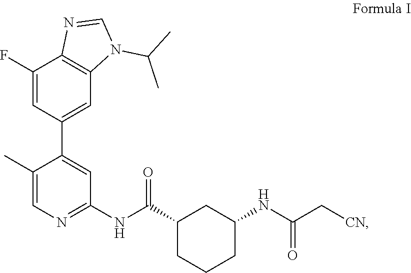 [SMILES: Cc1cnc(NC(=O)[C@H]2CCC[C@@H](NC(=O)CC#N)C2)cc1-c1cc(F)c2ncn(C(C)C)c2c1]

The synthesis of the compound of Formula (I) is set forth in U.S. Pat. No. 11,673,893. Pharmaceutically acceptable salts of the compound of Formula (I) are described in published international application WO 2023/064920.

BTK inhibitors and BCL2 inhibitors have shown clinical efficacy in lymphoid malignancies. BTK inhibitors are approved for the treatment of diffuse large B-cell lymphoma (DLBCL), chronic lymphocytic leukemia (CLL) and mantle cell lymphoma (MCL), whereas BCL2 inhibitors are approved for the treatment of CLL. However, despite strong clinical responses to both classes of agents, resistance rapidly develops.

Accordingly, there is a need in the art for specific inhibitors of CDK9 and treatment regimen for use in relapse/refractory lymphoid malignancies. The present disclosure addresses this need.

### SUMMARY

The disclosure provides methods of treating cancer in a subject in need thereof, comprising administering to the subject a treatment regimen comprising:
(a) a compound of Formula (I):

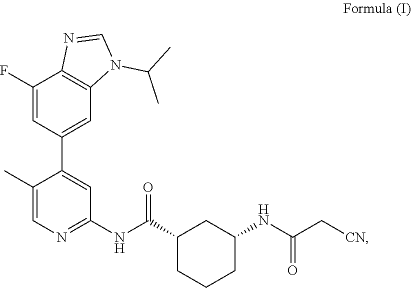 [SMILES: Cc1cnc(NC(=O)[C@H]2CCC[C@@H](NC(=O)CC#N)C2)cc1-c1cc(F)c2ncn(C(C)C)c2c1]

or a pharmaceutically acceptable salt thereof; and
(b) a second compound that is a BTK inhibitor or a BCL2 inhibitor.

### BRIEF DESCRIPTION OF THE DRAWINGS

In the drawings, which are not necessarily drawn to scale, like numerals can describe similar components in different views. Like numerals having different letter suffixes can represent different instances of similar components. The drawings illustrate generally, by way of example, but not by way of limitation, various aspects discussed in the present document. In the drawings:

FIG. 1 shows a waterfall plot of a CellTiter-Glo (CTG) assay IC50s assessing growth inhibition potency in DLBCL and CLL cell lines transiently treated (4 hour) with the compound of Formula (I). Cell viability was assessed after 48 hours.

FIG. 2 shows results from a caspase 3/7-Glo assay demonstrating induction of apoptosis in DLBCL and CLL cell lines after 6 h treatment with the compound of Formula (I).

FIG. 3 shows results from a cell line-derived xenograft (CDX) DLBCL models treated with the compound of Formula (I) intravenously (QW, BID q2h) show significant inhibition of tumor growth. Data represented as mean±SEM. N=8, ***P<0.001, ****P<0.0001 by Mann-Whitney U test.

FIG. 4 shows results from a caspase 3/7-Glo assay assessing induction of apoptosis by the compound of Formula (I) in DLBCL cell lines, with and without overnight pre-treatment with Zanubrutinib (1 nM).

FIG. 5 shows TMD-8 (DLBCL) CDX demonstrating potent inhibition of tumor growth by the compound of Formula (I) (7.5 mg/kg, QW, BID q2h) administered intravenously, in monotherapy and in combination with Zanubrutinib (5 mg/kg, BID) or Pirtobrutinib (15 mg/kg, BID), administered orally.

FIG. 6 shows results from intravenously administered the compound of Formula (I) (30 mg/kg, QW, BID q2h) alone and in combination with Zanubrutinib (25 mg/kg, QD), administered orally, potently inhibits tumor growth in LY2264 and LY2298 (DLBCL) patient-derived xenografts (PDX); 100% complete response (mean tumor volume=0 mm3) observed in combination group. Data represented as mean±SEM. N=8, *P<0.05, **P<0.001, ***P<0.001 by Mann-Whitney U test.

FIG. 7 shows a Western blot assessing modulation of MCL1, BFL1, BMF and Cleaved Caspase 3 in TMD8 cells following treatment with the compound of Formula (I)±Zanubrutinib pre-treatment, and a Western blot showing decrease in MCL1 and BMF in LY2264 tumors following the compound of Formula (I) and Zanubrutinib treatment in vivo.

FIG. 8A depicts a caspase 3/7-Glo assay assessing induction of apoptosis in DLBCL cells co-treated with the compound of Formula (I) and varying doses of Venetoclax for 6 h.

FIG. 8B depicts a caspase 3/7-Glo assay assessing induction of apoptosis in CLL cells co-treated with the compound of Formula (I) and varying doses of Venetoclax for 6 h.

FIG. 9 shows TMD-8 (DLBCL) and U-2932 (DLBCL) CDX demonstrating potent inhibition of tumor growth by the compound of Formula (I) (7.5 mg/kg, QW BID q2h) administered intravenously, in monotherapy and in combination with Venetoclax (50 mg/kg, QD) administered orally. Data represented as mean±SEM. N=8, *P<0.05, **P<0.001, ***P<0.001 by Mann-Whitney U test.

FIGS. 10 and 11 depict Caspase 3/7 activity assay showing induction of apoptosis in PBMCs derived from patients with untreated or relapsed/refractory (R/R) CLL, following 6 h treatment with the compound of Formula (I).

FIG. 12 depicts caspase 3/7 activity assay showing induction of apoptosis by the compound of Formula (I) in CLL PBMCs, with and without overnight pre-treatment with varying doses of BTKi.

FIG. 13 depicts caspase 3/7 activity assay showing induction of apoptosis in CLL PBMCs co-treated with the compound of Formula (I) and varying doses of Venetoclax.

FIG. 14 shows activity of the compound of Formula (I) in Ibrutinib sensitive and resistant MCL cell lines in vitro in a CTG assay.

FIG. 15 shows that the compound of Formula (I) (i.v. 15 mg/kg, QW BID q2h) shows significant tumor growth inhibition in the Mino CDX model of Ibrutinib resistant MCL. Anti-tumor activity remains similar to the parental Mino model. Ibrutinib-resistance was induced through repeat in vivo passage and treatment with Ibrutinib. Data represented as mean±SEM. N=7, *P<0.05, ****P<0.0001 by Mann-Whitney U test.

### DETAILED DESCRIPTION OF ILLUSTRATIVE EMBODIMENTS

The present disclosure may be understood more readily by reference to the following detailed description of desired embodiments and the examples included therein.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing. All publications, patent applications, patents and other references mentioned herein are incorporated by reference in their entirety. The materials, methods, and examples disclosed herein are illustrative only and not intended to be limiting.

The singular forms “a,” “an,” and “the” include plural referents unless the context clearly dictates otherwise.

As used in the specification and in the claims, the term “comprising” can include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any impurities that might result therefrom, and excludes other ingredients/steps.

As used herein, the terms “about” and “at or about” mean that the amount or value in question can be the value designated some other value approximately or about the same. It is generally understood, as used herein, that it is the nominal value indicated±10% variation unless otherwise indicated or implied. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Unless indicated to the contrary, the numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently of the endpoints. The endpoints of the ranges and any values disclosed herein are not limited to the precise range or value; they are sufficiently imprecise to include values approximating these ranges and/or values.

The term “administering,” when used in the context of administering a therapeutic agent to a subject, refers to introducing the therapeutic agent into the subject's body. For example, therapeutic agents may be introduced into a subject's body orally, nasally, subcutaneously, intravenously, intravesically, intramuscularly, transdermally, vaginally, rectally or in any combination thereof.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The term “subject” is used herein to describe an animal, for example, a mammal, to whom treatment with the treatment regimen according to the disclosure is provided. In some aspects, the mammal is a human to whom treatment is provided. In other aspects, the mammal is a non-human to whom treatment is provided.

“Pharmaceutically acceptable” means approved or approvable by a regulatory agency of the United States Federal government or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethane-sulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

As used herein, the term “treatment regimen” refers to coordinated dosages and administration timings of each of Formula (I) (or pharmaceutically acceptable salt thereof) and the second compound that is a BTK inhibitor or a BCL2 inhibitor, such that Formula (I) (or pharmaceutically acceptable salt thereof) and the second compound that is a BTK inhibitor or a BCL2 inhibitor are administered to the subject during an overlapping period of time. Thus, the “treatment regimen” of the disclosure encompasses both concurrent administration of the compound of Formula (I) (or a pharmaceutically acceptable salt thereof) and the second compound that is a BTK inhibitor or a BCL2 inhibitor; and sequential administration of the compound of Formula (I) (or a pharmaceutically acceptable salt thereof) and the second compound that is a BTK inhibitor or a BCL2 inhibitor. Moreover, the treatment regimen encompasses administration of the compound of Formula (I) (or a pharmaceutically acceptable salt thereof) and the second compound that is a BTK inhibitor or a BCL2 inhibitor on the same day; on different days; and any permutation thereof.

In some aspects, the disclosure is directed to methods of treating cancer in a subject in need thereof, comprising administering to the subject a treatment regimen comprising:
(a) a compound of Formula (I):

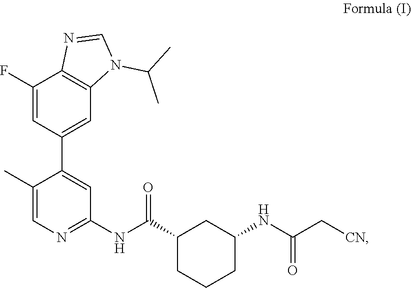 [SMILES: Cc1cnc(NC(=O)[C@H]2CCC[C@@H](NC(=O)CC#N)C2)cc1-c1cc(F)c2ncn(C(C)C)c2c1]

or a pharmaceutically acceptable salt thereof; and
(b) a second compound that is a BTK inhibitor or a BCL2 inhibitor.

In some aspects of the disclosed methods, the subject is administered a compound of Formula (I).

In other aspects of the disclosed methods, the subject is administered a pharmaceutically acceptable salt of a compound of Formula (I).

In some embodiments of the disclosed methods, the compound of Formula (I), or a pharmaceutically acceptable salt thereof, and the second compound that is a BTK inhibitor or a BCL2 inhibitor are administered concurrently or sequentially.

In some embodiments of the disclosed methods, the compound of Formula (I), or a pharmaceutically acceptable salt thereof, and the second compound that is a BTK inhibitor or a BCL2 inhibitor are administered concurrently.

In other embodiments of the disclosed methods, the compound of Formula (I), or a pharmaceutically acceptable salt thereof, and the second compound that is a BTK inhibitor or a BCL2 inhibitor are administered sequentially.

In some aspects, the subject is administered the compound of Formula (I), or a pharmaceutically acceptable salt of a compound of Formula (I) (on a Formula (I) basis), in an amount of about 5 mg/m²-about 35 mg/m² per dose, such as, for example, about 5 mg/m², about 6 mg/m², about 7 mg/m², about 8 mg/m², about 9 mg/m², about 10 mg/m², about 11 mg/m², about 12 mg/m², about 13 mg/m², about 14 mg/m², about 15 mg/m², about 16 mg/m², about 17 mg/m², about 18 mg/m², about 19 mg/m², about 20 mg/m², about 21 mg/m², about 22 mg/m², about 23 mg/m², about 24 mg/m², about 25 mg/m², about 26 mg/m², about 27 mg/m², about 28 mg/m², about 29 mg/m², about 30 mg/m², about 31 mg/m², about 32 mg/m², about 33 mg/m², about 34 mg/m², or about 35 mg/m² per dose. In some embodiments, the subject is administered the compound of Formula (I), or a pharmaceutically acceptable salt of a compound of Formula (I) (on a Formula (I) basis), in an amount of about 9 mg/m²-about 24 mg/m² per dose.

In some embodiments, the subject is administered the compound of Formula (I), or a pharmaceutically acceptable salt of a compound of Formula (I) (on a Formula (I) basis), in an amount of 5 mg/m²-35 mg/m² per dose, such as, for example, 5 mg/m², 6 mg/m², 7 mg/m², 8 mg/m², 9 mg/m², 10 mg/m², 11 mg/m², 12 mg/m², 13 mg/m², 14 mg/m², 15 mg/m², 16 mg/m², 17 mg/m², 18 mg/m², 19 mg/m², 20 mg/m², 21 mg/m², 22 mg/m², 23 mg/m², 24 mg/m², 25 mg/m², 26 mg/m², 27 mg/m², 28 mg/m², 29 mg/m², 30 mg/m², 31 mg/m², 32 mg/m², 33 mg/m², 34 mg/m², or 35 mg/m² per dose. In some embodiments, the subject is administered the compound of Formula (I), or a pharmaceutically acceptable salt of a compound of Formula (I) (on a Formula (I) basis), in an amount of 9 mg/m²-24 mg/m² per dose.

In some aspects, the compound of Formula (I), or pharmaceutically acceptable salt of a compound of Formula (I), is administered intravenously.

In some embodiments of the disclosed methods, the second compound is a BTK inhibitor.

In some embodiments of the disclosed methods, the BTK inhibitor is ibrutinib, acalabrutinib, zanubrutinib, or pirtobrutinib.

In some embodiments of the disclosed methods, the BTK inhibitor is ibrutinib. Ibrutinib is marketed under the tradename Imbruvica®.

In some embodiments, the ibrutinib is administered orally.

In some embodiments, the ibrutinib is administered in an amount of about 420 mg per day.

In other embodiments, the ibrutinib is administered in an amount of about 240 mg/m² per day.

In some embodiments, the ibrutinib is administered in accordance with the December 2024 Revision (Janssen Biotech, Inc.) of the Imbruvica® prescribing information.

In some embodiments of the disclosed methods, the BTK inhibitor is acalabrutinib. Acalabrutinib is marketed under the tradename Calquence®.

In some embodiments, the acalabrutinib is administered orally.

In some embodiments, the acalabrutinib is administered in an amount of about 100 mg twice per day.

In some embodiments, the acalabrutinib is administered in accordance with the January 2025 Revision (AstraZeneca Pharmaceuticals LP) of the Calquence® prescribing information.

In some embodiments of the disclosed methods, the BTK inhibitor is zanubrutinib. Zanubrutinib is marketed under the tradename Brukinsa®.

In some embodiments, the zanubrutinib is administered orally.

In some embodiments, the zanubrutinib is administered in an amount of about 160 mg twice per day. In other embodiments, the zanubrutinib is administered in an amount of about 320 mg once per day.

In some embodiments, the zanubrutinib is administered in accordance with the January 2025 Revision (BeiGene USA, Inc.) of the Brukinsa® prescribing information.

In some embodiments of the disclosed methods, the BTK inhibitor is pirtobrutinib. Pirtobrutinib is marketed under the tradename Jaypirca®.

In some embodiments, the pirtobrutinib is administered orally.

In some embodiments, the pirtobrutinib is administered in an amount of about 200 mg once per day.

In some embodiments, the pirtobrutinib is administered in accordance with the June 2024 Revision (Eli Lilly and Company) of the Jaypirca® prescribing information.

In some aspects of the disclosed methods, the second compound is a BCL2 inhibitor.

In some embodiments of the disclosed methods, the BCL2 inhibitor is venetoclax. Venetoclax is marketed under the tradename Venclexta®.

In some embodiments, the venetoclax is administered orally.

In some embodiments, the venetoclax is administered in an amount of about 20 mg to about 600 mg per day. In some embodiments, the venetoclax is administered in an amount of about 400 mg per day.

In some embodiments, the venetoclax is administered in accordance with the July 2024 Revision (Abb Vie Inc.) of the Venclexta® prescribing information.

In some aspects, the methods of the disclosure are directed to treating cancer in a subject. In some embodiments, the cancer is a lymphoid malignancy.

In some embodiments, the lymphoid malignancy is Diffuse Large B Cell Lymphoma (DLBCL), Mantle Cell Lymphoma (MCL), or Chronic Lymphocytic Leukemia (CLL).

In some embodiments, the lymphoid malignancy is Diffuse Large B Cell Lymphoma (DLBCL).

In some embodiments, the lymphoid malignancy is Mantle Cell Lymphoma (MCL).

In some embodiments, the lymphoid malignancy is Chronic Lymphocytic Leukemia (CLL).

In some embodiments, the cancer is a relapsed or refractory malignancy.

In some embodiments, the subject is a human subject. In some embodiments, the subject is a mammalian subject, for example, a human, canine, equine, bovine, murine, rattus, ovine, feline, non-human primate, and the like.

The disclosure is also directed to the following aspects 1-6:
Aspect 1. A method of treating a transcriptionally active cancer in a subject in need thereof, comprising administering to the subject a therapeutically effective amount of a CDK9 inhibitor.
Aspect 2. The method of aspect 1, wherein the CDK9 inhibitor comprises Compound I, or a pharmaceutically acceptable salt thereof.
Aspect 3. The method of any one of the preceding aspects, wherein the CDK9 inhibitor is administered using a dosing regimen and schedule disclosed herein.
Aspect 4. The method of any one of the preceding aspects, wherein the transcriptionally active cancer comprises one or more lymphoid malignancies.
Aspect 5. The method of any one of the preceding aspects, wherein the one or more lymphoid malignancies comprise Diffuse Large B Cell Lymphoma (DLBCL), Mantle Cell Lymphoma (MCL) and Chronic Lymphocytic Leukemia (CLL).
Aspect 6. The method of any one of the preceding aspects, wherein the CDK9 inhibitor is administered in combination with one or more BTK inhibitors (BTKi) or BCL2 inhibitors (BCL2i).

### EXAMPLES

### Example 1. Treatment of DLBCL and CLL Models with Formula I

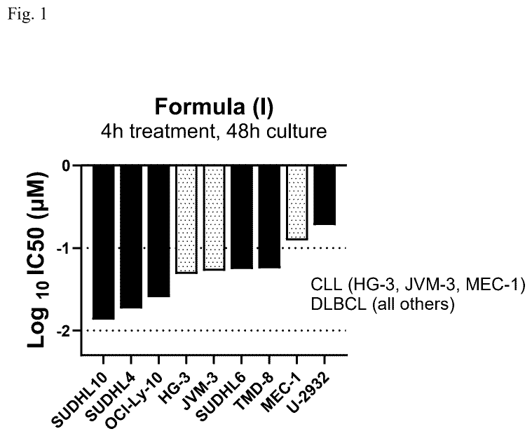

CellTiter-Glo (CTG) assay (Promega, G9241): DLBCL cell lines (SUDHL10, SUDHL4, OCI-Ly-10, SUDHL6, TMD-8, and U-2932) and CLL cell lines (HG-3, JVM-3, and MEC-1) were transiently treated (4 h) with Formula I. Cell viability was assessed after 48 h according to manufacturer instructions. See FIG. 1.

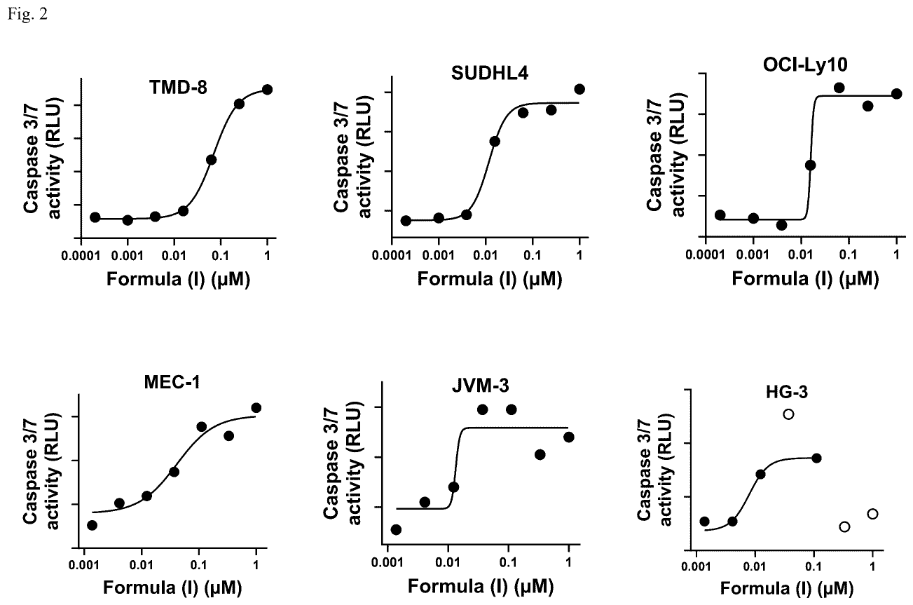

A Caspase 3/7-Glo assay (Promega, G8090) was used to demonstrate induction of apoptosis in DLBCL (SUDHL4, OCI-Ly-10, and TMD-8) and CLL (HG-3, JVM-3, and MEC-1) cell lines after 6 h treatment with Formula I. See FIG. 2.

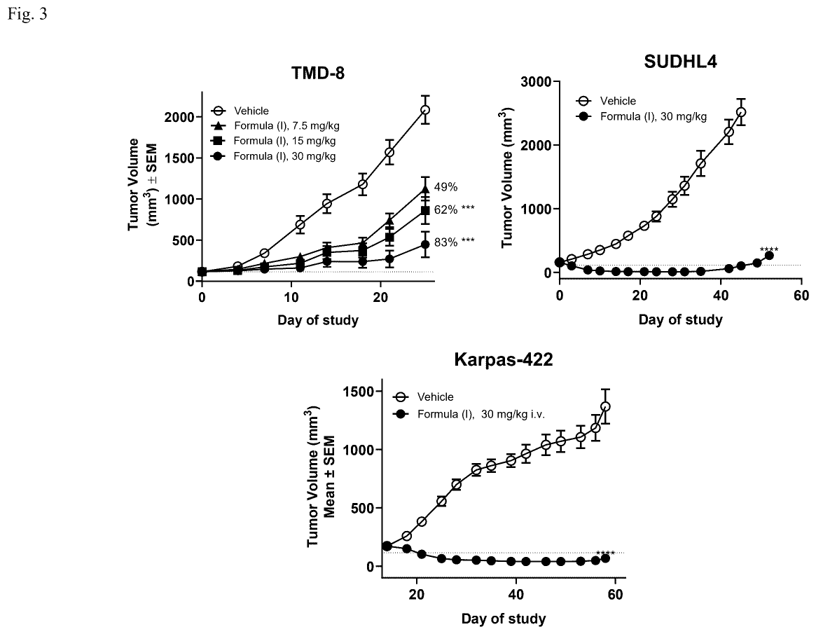

Cell line-derived xenograft (CDX) DLBCL models were treated with Formula (I) intravenously (QW, BID q2h) and showed significant inhibition of tumor growth. In TMD-8 subcutaneous xenograft, a 49% tumor growth inhibition was observed at Formula (I) dose of 7.5 mg/kg; a 62% tumor growth inhibition was observed at Formula (I) dose of 15 mg/kg; and an 83% tumor growth inhibition was observed at Formula (I) dose of 30 mg/kg. In SUDHL4 subcutaneous xenograft, a 125% tumor growth inhibition was observed at Formula (I) dose of 30 mg/kg. In Karpas-422 subcutaneous xenograft, a 108% tumor growth inhibition was observed at Formula (I) dose of 30 mg/kg. See FIG. 3 (Data represented as mean±SEM. N=8, ***P<0.001, ****P<0.0001 by Mann-Whitney U test).

These results demonstrate that short-term treatment with the compound of Formula (I) is efficacious in DLBCL and CLL models.

### Example 2. Combination with BTK Inhibitors

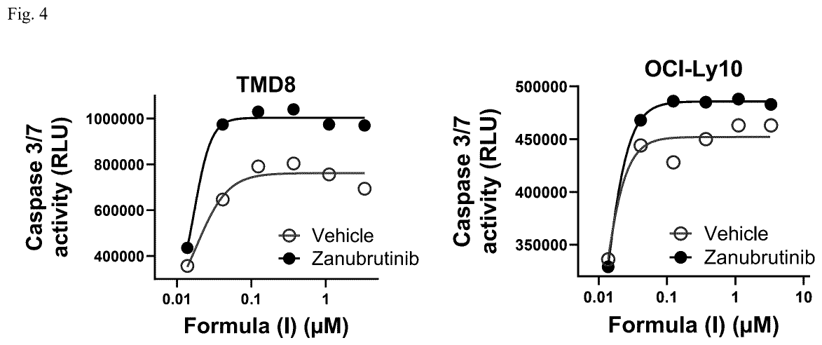

A Caspase 3/7-Glo assay (Promega, G8090) was used to assess induction of apoptosis by Formula (I) in DLBCL cell lines (TMD-8 and OCI-Ly-10) with and without overnight pre-treatment with Zanubrutinib (1 nM). See FIG. 4.

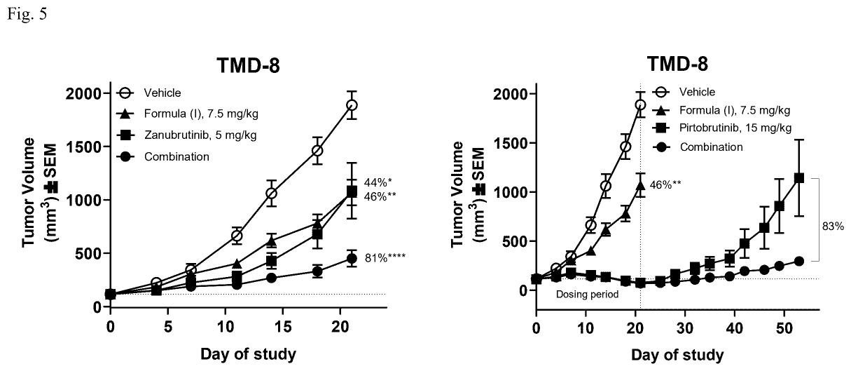

Formula (I) was administered intravenously (7.5 mg/kg, BID q2h QW) as monotherapy or in combination with Zanubrutinib (5 mg/kg, BID administered orally) or Pirtobrutinib (15 mg/kg, BID administered orally) to TMD-8 (DLBCL) cell line-derived xenograft (CDX). This experiment demonstrated potent inhibition of tumor growth. Formula (I) monotherapy (7.5 mg/kg, BID q2h QW) resulted in 46% tumor growth inhibition. Zanubrutinib (5 mg/kg, BID administered orally) resulted in 44% tumor growth inhibition. The combination of Formula (I) (7.5 mg/kg, BID q2h QW) and Zanubrutinib (5 mg/kg, BID administered orally) resulted in 81% tumor growth inhibition. The combination of Formula (I) (7.5 mg/kg, BID q2h QW) and Pirtobrutinib (15 mg/kg, BID administered orally) resulted in 83% tumor growth inhibition. See FIG. 5.

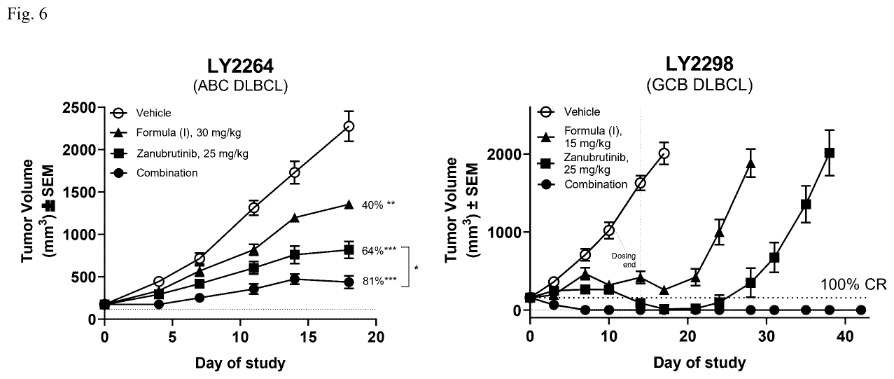

Intravenously administered Formula (I) (30 mg/kg, QW, BID q2h) alone or in combination with Zanubrutinib (25 mg/kg, QD), administered orally, potently inhibits tumor growth in LY2264 and LY2298 (DLBCL) patient-derived xenografts (PDX). In LY2264 (DLBCL) patient-derived xenografts (PDX), Formula (I) alone showed 40% tumor growth inhibition; Zanubrutinib alone showed 64% tumor growth inhibition; the combination of Formula (I) and Zanubrutinib showed 81% tumor growth inhibition. See FIG. 6 (Data represented as mean±SEM. N=8, *P<0.05, **P<0.001, ***P<0.001 by Mann-Whitney U test).

In LY2298 (DLBCL) patient-derived xenografts (PDX), the combination of Formula (I) (15 mg/kg, QW, BID q2h) and Zanubrutinib (25 mg/kg, QD) showed 100% complete response (mean tumor volume=0 mm3). See FIG. 6.

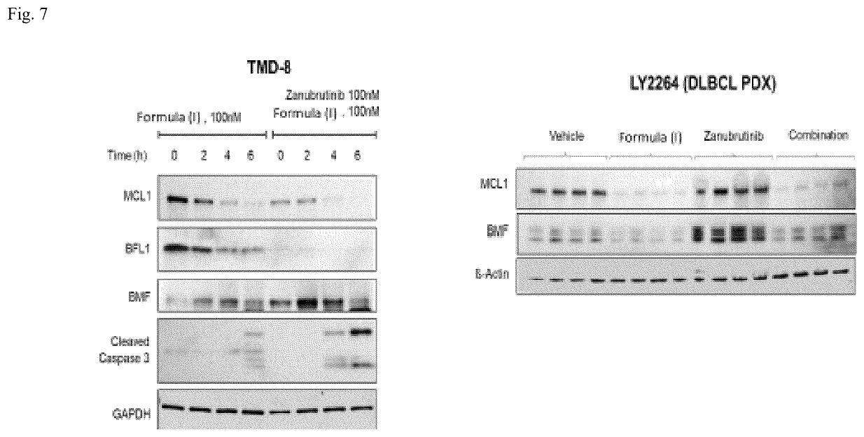

Western blot was used to assess modulation of MCL1, BFL1, BMF and Cleaved Caspase 3 in TMD8 cells following treatment with Formula (I) with or without Zanubrutinib pre-treatment. Western blot was used to show the decrease in MCL1 and BMF in LY2264 tumors following Formula (I) and Zanubrutinib treatment in vivo. See FIG. 7.

### Example 3. Combination with Venetoclax

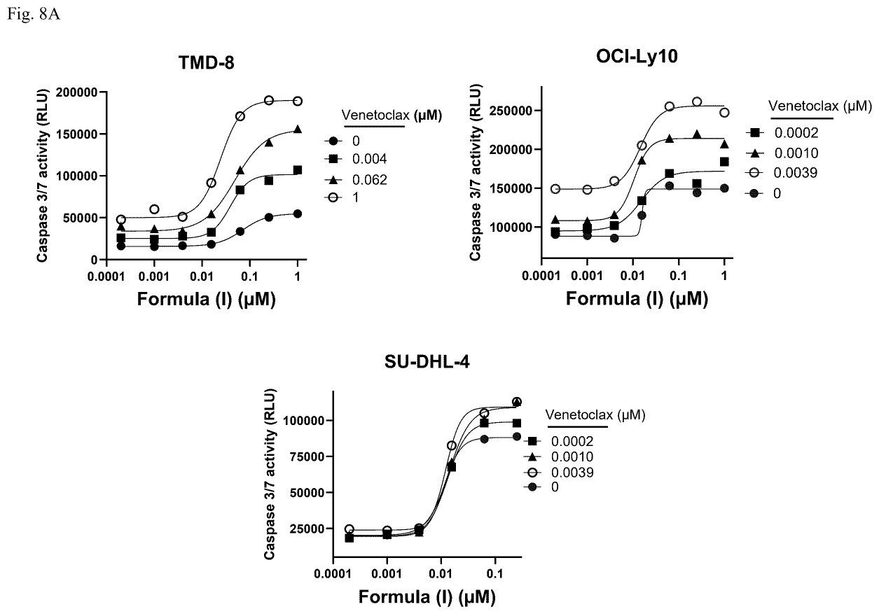

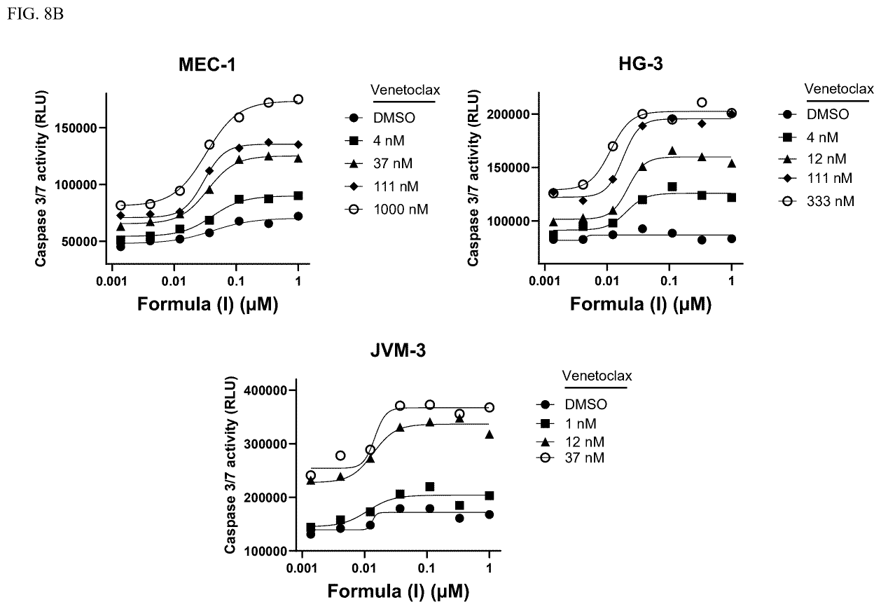

A Caspase 3/7-Glo assay was used to assess induction of apoptosis in DLBCL cells (SUDHL4, OCI-Ly-10, TMD-8) and CLL cells (HG-3, JVM-3, and MEC-1) co-treated with Formula (I) and varying doses of Venetoclax for 6 hours. DLBCL cell lines (SUDHL10, SUDHL4, OCI-Ly-10, SUDHL6, TMD-8, and U-2932) and CLL cell lines (HG-3, JVM-3, and MEC-1). See FIGS. 8A and 8B.

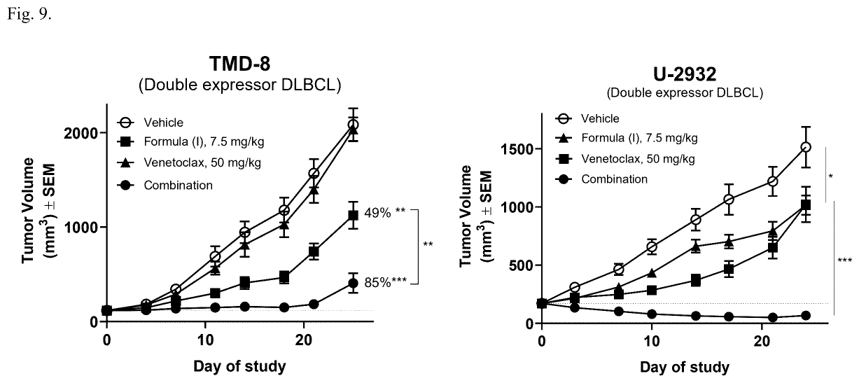

TMD-8 (DLBCL) and U-2932 (DLBCL) CDXs were used to demonstrate potent inhibition of tumor growth by Formula (I) (7.5 mg/kg, QW BID q2h) administered intravenously, in monotherapy and in combination with Venetoclax (50 mg/kg, QD) administered orally. In the TMD-8 CDX, Formula (I) (7.5 mg/kg, QW BID q2h) administered intravenously as monotherapy showed 49% tumor growth inhibition; Venetoclax (50 mg/kg, QD) administered orally showed no tumor growth inhibition; and the combination of Formula (I) (7.5 mg/kg, QW BID q2h) administered intravenously and Venetoclax (50 mg/kg, QD) administered orally showed 85% tumor growth inhibition. See FIG. 9 (Data represented as mean±SEM. N=8, *P<0.05, **P<0.001, ***P<0.001 by Mann-Whitney U test).

In the U-2932 CDX, Formula (I) (7.5 mg/kg, QW BID q2h) administered intravenously as monotherapy showed 37% tumor growth inhibition; Venetoclax (50 mg/kg, QD) administered orally showed 37% tumor growth inhibition; and the combination of Formula (I) (7.5 mg/kg, QW BID q2h) administered intravenously and Venetoclax (50 mg/kg, QD) administered orally showed 107% tumor growth inhibition. See FIG. 9 (Data represented as mean±SEM. N=8, *P<0.05, **P<0.001, ***P<0.001 by Mann-Whitney U test).

### Example 4. Treatment of Relapsed/Refractory (R/R) Cancer

Formula (I) is efficacious in treatment-naïve and relapsed/refractory (R/R) primary CLL ex vivo as monotherapy and in combination with BTKi or Venetoclax.

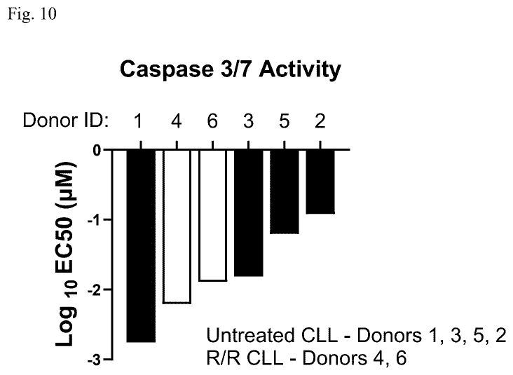

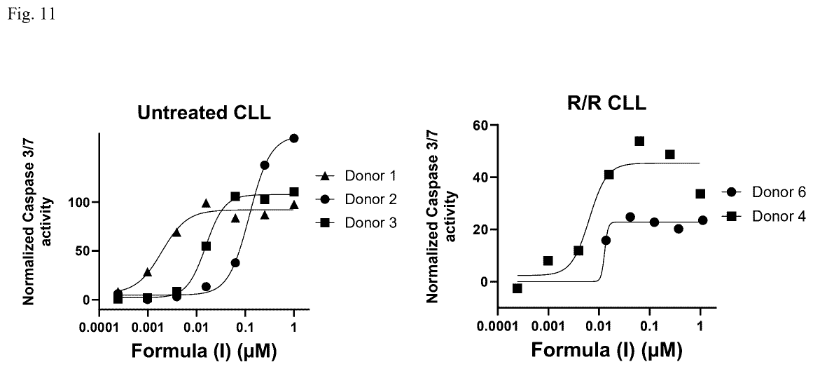

A Caspase-Glo® 3/7 activity assay was used to show induction of apoptosis in PBMCs derived from patients with untreated or relapsed/refractory (R/R) CLL, following 6 h treatment with Formula I. See FIGS. 10, 11.

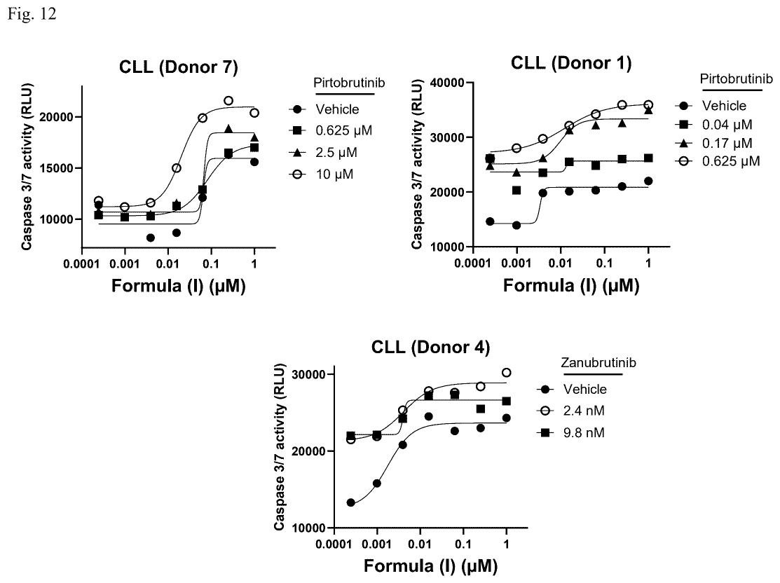

A Caspase 3/7 activity assay was used to show induction of apoptosis by Formula (I) in CLL PBMCs, with and without overnight pre-treatment with varying doses of BTKi. See FIG. 12.

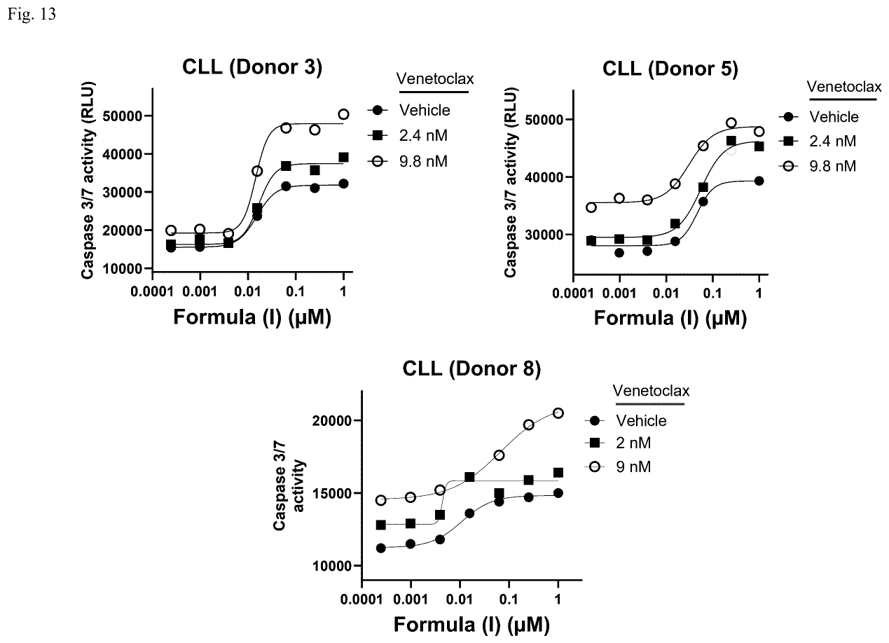

A Caspase 3/7 activity assay was used to show induction of apoptosis in CLL PBMCs co-treated with Formula (I) and varying doses of Venetoclax. See FIG. 13.

### Example 5. Formula (I) Overcomes Ibrutinib Resistance in Mantle Cell Lymphoma

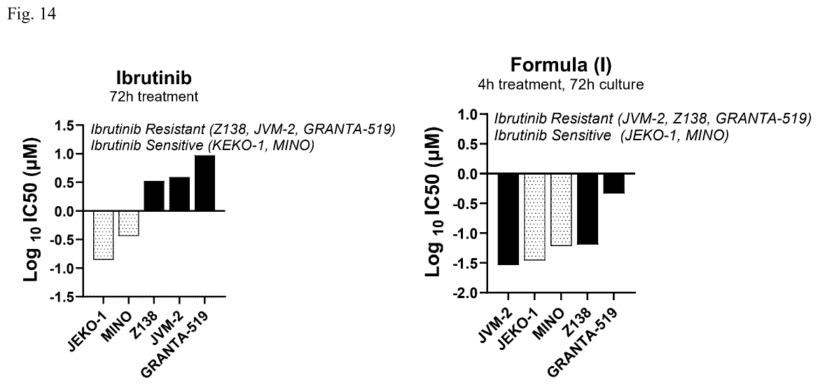

A CTG assay was used to demonstrate that Formula (I) shows activity in Ibrutinib-sensitive and Ibrutinib-resistant MCL cell lines in vitro. See FIG. 14.

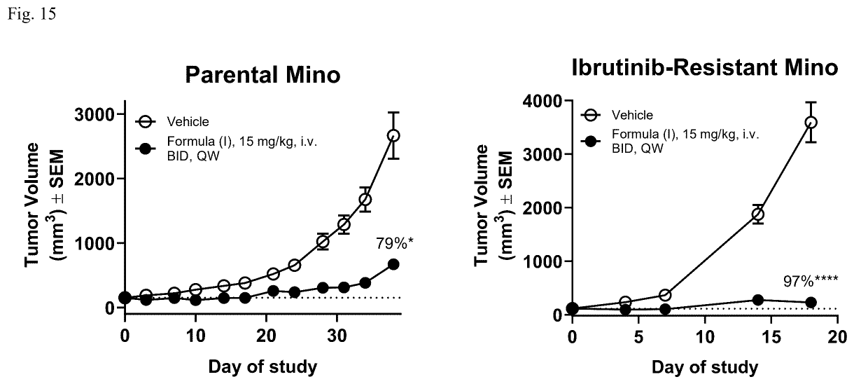

Formula (I) administered (i.v. 15 mg/kg, QW BID q2h) intravenously in the Mino CDX model of Ibrutinib resistant MCL showed significant tumor growth inhibition. Anti-tumor activity remains similar to the parental Mino model. Ibrutinib-resistance was induced through repeat in vivo passage and treatment with Ibrutinib. See FIG. 15 (Data represented as mean±SEM. N=7, *P<0.05, ****P<0.0001 by Mann-Whitney U test).

The results described above demonstrate that the compound of Formula (I) is efficacious in pre-clinical models of DLBCL, CLL and MCL. Furthermore, the compound of Formula I, when combined with BCL2i and BTKi potently induces apoptosis in vitro and inhibit tumor growth (including regressions), in vivo. The compound of Formula (I) is also shown to potently induce apoptosis in treatment-naïve and R/R primary CLL ex vivo. As demonstrated, the compound of Formula (I) overcomes resistance to Ibrutinib in MCL in vitro and in vivo.

What is claimed is:

1. A method of treating cancer in a subject in need thereof, comprising administering to the subject a treatment regimen comprising:
(a) a compound of Formula (I): 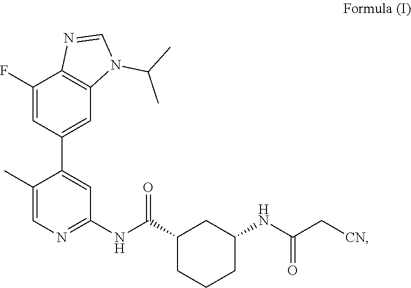 [SMILES: Cc1cnc(NC(=O)[C@H]2CCC[C@@H](NC(=O)CC#N)C2)cc1-c1cc(F)c2ncn(C(C)C)c2c1]
or a pharmaceutically acceptable salt thereof; and
(b) a second compound that is a BTK inhibitor or a BCL2 inhibitor.

2. The method of claim 1, wherein the compound of Formula (I), or a pharmaceutically acceptable salt thereof, and the second compound are administered concurrently or sequentially.

3. The method of claim 1, wherein the second compound is a BTK inhibitor.

4. The method of claim 3, wherein the BTK inhibitor is ibrutinib, acalabrutinib, zanubrutinib, or pirtobrutinib.

5. The method of claim 4, wherein the BTK inhibitor is ibrutinib.

6. The method of claim 4, wherein the BTK inhibitor is acalabrutinib.

7. The method of claim 4, wherein the BTK inhibitor is zanubrutinib.

8. The method of claim 4, wherein the BTK inhibitor is pirtobrutinib.

9. The method of claim 1, wherein the second compound is a BCL2 inhibitor.

10. The method of claim 9, wherein the BCL2 inhibitor is venetoclax.

11. The method of claim 1, wherein the subject is administered the compound of Formula (I), or a pharmaceutically acceptable salt of a compound of Formula (I) (on a Formula (I) basis), in an amount of about 9 mg-about 24 mg per dose.

12. The method of claim 1, wherein the compound of Formula (I), or pharmaceutically acceptable salt of a compound of Formula (I), is administered intravenously.

13. The method of claim 1, wherein the cancer is a lymphoid malignancy.

14. The method of 13, wherein the lymphoid malignancy is Diffuse Large B Cell Lymphoma (DLBCL), Mantle Cell Lymphoma (MCL) or Chronic Lymphocytic Leukemia (CLL).

15. The method of claim 1, wherein the cancer is a relapsed or refractory cancer.
